(19) United States
(12) Patent Application Publication
(10) Pub. No.: US 2026/0355934 A1
(43) Pub. Date: Nov. 12, 2026

(54) ORTHOPAEDIC DEVICE

(71) Applicant: OTTO BOCK HEALTHCARE PRODUCTS GMBH, Wien (AT)

(72) Inventor: Wolfgang SAUBERER, Wien (AT)

(21) Appl. No.: 19/125,153

(22) PCT Filed: Nov. 7, 2023
(86) PCT No.: PCT/EP2023/080951

§ 371 Date: Apr. 28, 2025

(30) Foreign Application Priority Data
Nov. 18, 2022 (DE) 10 2022 130 585.6

(51) Int. Cl. B25J 9/00; A61F 5/01

(52) U.S. Cl. CPC B25J 9/0006; A61F 5/0125

(57) ABSTRACT

The invention relates to an orthopaedic device having an upper part (100) and a lower part (200) which is mounted thereon in a displaceable manner and is coupled to an actuator (30) that displaces the lower part relative to the upper part, wherein the actuator has an electric motor (40), an energy store (50) and a control device (60), wherein a first changeover switch (62) is arranged between the energy store and the electric motor, which changeover switch is coupled to the control device and switches the supply voltage (Us) of a motor control (66) of the electric motor between the starting voltage (Uo) of the energy store and a voltage converter (80) with a supply voltage that has changed with respect to the starting voltage.

The invention relates to an orthopedic device having an upper part and a lower part, which is mounted displaceably to the latter and is coupled to an actuator that displaces the lower part relative to the upper part, the actuator comprising an electric motor, an energy store and a control device.

Orthopedic devices are in particular prostheses, orthoses and exoskeletons. Prostheses replace limbs or body parts which are not present, or no longer present, as far as possible in respect of their shape and/or function. The simplest prostheses have a purely cosmetic function or fully replace a member, for example by a distal phalanx of a finger being replaced. In more complex prosthetic therapies, several prosthesis components are connected and fastened to one another, in particular connected pivotably to one another by joints.

In order to influence relative movements of the prosthesis components, blocking devices have been developed with which it is possible to lock and unlock an extended prosthetic knee joint so that the user of the prosthesis can sit down. Drive devices have been developed in order to transmit movements in the shoulder to prosthetic hands via cables, so that objects can be gripped and held. In order to influence a movement profile, dampers, in particular hydraulic dampers, as well as energy stores, particularly in the form of springs, have been arranged on the individual components. The aim, inter alia, is to achieve optimal functionality and where appropriate to allow a movement sequence that is as natural as possible.

In order to assist or brake movements of prosthesis components, drives have been integrated into the prosthesis so as to create active prostheses. There are also corresponding embodiments in passive and active orthoses or exoskeletons which are arranged on existing members. Cross-joint orthoses and exoskeletons may likewise be equipped with springs, force stores and/or motor drives.

In order to control both passive and active prostheses, orthoses and exoskeletons, control devices are arranged on orthopedic devices, which generate control signals on the basis of sensor values or stored sequences in order to activate, deactivate or modulate drives or in order to modify resistances, for example by opening or closing valves, releasing or blocking stored energy from energy stores, switching on, switching off or modulating electric motors, or the like.

In active orthopedic devices with electric motor drives, the energy store generally consists of a battery or an accumulator. In an electric motor drive, there is a proportionality between the required supply voltage and the rotational speed of the drive, as well as a proportionality between the torque of the drive and its motor current. The maximum values of current and voltage of the energy supply therefore determine the maximum torque as well as the maximum rotational speed of the drive.

In order to be able to provide both high torques and high rotational speeds for driving an orthopedic device, correspondingly large energy stores or electric motors need to be provided. Since this increases the weight of the orthopedic device, limits are set on increasing the size of the energy stores and/or motors. An electric motor is an electromechanical converter which transforms electrical energy into mechanical energy. An electric motor means in particular DC motors, AC motors, piezoelectric actuators, motors that function using the Lorentz force, reluctance motors and thermoelectric actuators.

It is an object of the present invention to provide an orthopedic device which functions with a comparatively lightweight energy store, the drive of which has a comparatively large working range.

This object is achieved by an orthopedic device having the features of the main claim. Advantageous embodiments and developments of the invention are disclosed in the dependent claims, the description and the figures.

The orthopedic device having an upper part and a lower part, which is mounted displaceably to the latter and is coupled to an actuator that displaces the lower part relative to the upper part, the actuator comprising an electric motor, an energy store and a control device, is one wherein a first selector switch is arranged between the energy store and the electric motor and is coupled to the control device, and switches the supply voltage of a motor controller of the electric motor between the output voltage of the energy store and a voltage converter with a supply voltage that is modified, in particular increased, in relation to the initial setting. The selector switch, which is arranged in the control device and supplies the motor controller either directly by the energy store with its output voltage or with an increased voltage, is arranged between the motor controller and the energy store. Besides a certain rapidity of the adjustment, active orthopedic devices also need to apply a sufficient torque in order to be able to perform the desired movements or actions or in order to be able to influence a movement situation in the intended way. Rapid movements are often to be performed with a low torque without high loading, while movements that require a high torque are generally performed slowly. With the control device and the selector switch, in a first switch state it is possible to conduct the output voltage directly to the electric motor so that a high current is supplied to the motor with a comparatively low supply voltage, which is equal to the output voltage of the energy store. It is therefore possible to make the electric motor generate a high torque. If a corresponding switching signal is output by the control device, the voltage converter is switched on or activated. The voltage converter modifies, in particular increases or reduces, the output voltage coming from the energy store and supplies the increased or reduced voltage as a supply voltage via the motor controller to the electric motor. The rotational speed or the possible rotational speed of the electric motor is thereby raised, or the maximum rotational speed of the electric motor is increased, concomitantly with a reduced maximum torque, or reduced with a correspondingly raised maximum torque. The direct interconnection of the motor controller with the energy store allows a high flow of current in the motor. The electric motor can thereby generate high torques at low rotational speeds. If higher rotational speeds are required, the voltage converter switches to an increased supply voltage. The control device in this case delivers the switching signal for adjusting the supply voltage. The control device is capable of detecting the increased voltage requirement, or a requirement for an increased torque of the motor, and of providing a corresponding supply voltage by switching the voltage converter. This is done, for example, by evaluating sensors that record movements, loads, states and/or positions, or modifications thereof, of prosthesis or orthosis components, of the environment and/or of the untreated contralateral side of the patient. The evaluation of the sensors may also take place in such a way that the further future behavior is inferred from the previous signal profiles.

In one embodiment, the output voltage of the voltage converter is dynamically adjustable in order to allow soft switching between the respectively provided or required supply voltages. In order to be able to cover the increased voltage requirement in individual phases of a step, demand-oriented adaptation of the output voltage of the voltage converter takes place. The demand-oriented adaptation minimizes the losses inside the voltage converter that occur because of the step-up conversion of the voltage. The dynamic adjustability allows soft switching of the supply voltage respectively provided.

The voltage converter is in one embodiment configured in such a way that it can convert both up and down, that is to say it can provide an increased or reduced voltage as the supply voltage. Alternatively, in one embodiment a step-up converter and a step-down converter are provided in the voltage converter in order to be able to provide the supply voltage respectively desired.

In one embodiment, the voltage converter is configured bidirectionally and has a step-up converter and a step-down converter or has only one converter, which can convert both up and down. In generator operation of the motor, when energy is being fed back into the battery, or into the energy store, the voltage fed back may therefore be adjusted. On the one hand, in forward operation the output voltage of the energy store may be raised to a higher level in order to supply the motor controller, and on the other hand the step-down converter allows backward operation. The electric motor operates with an increased supply voltage level in generator operation. The step-down conversion makes it possible to charge the electrical energy created in the motor back into the energy store in a controlled way. The generator operation allows a return flow into the energy store of electrical energy that is generated while the electric motor is operating at an increased voltage level of the supply voltage in generator operation. Particularly in the case of a dynamically adjustable voltage converter, it is therefore possible to reduce the losses in the step-up converter, or respectively the step-down converter, when the voltage of the motor is being converted up or down.

According to one development, a second selector switch for switching between the step-up converter and the step-down converter is coupled to the control device. The need for a second selector switch exists in particular when the output of the voltage converter cannot be deactivated. In such an embodiment of the voltage converter, the second selector switch always needs to be off when the first selector switch is on, and the second selector switch always needs to be on when the first selector switch is off.

In the initial setting of the first selector switch, the output voltage of the energy store is applied as a supply voltage to the motor controller of the electric motor, so that operation initially takes place with a low maximum rotational speed and a maximum torque. Only when there is a corresponding control signal, for example on the basis of sensors, is switching carried out to an increase of the supply voltage.

In one embodiment, the selector switch or the selector switches is/are configured as part of the voltage converter. The switching may therefore be partially or fully integrated in the voltage converter.

In one embodiment, the supply line of the motor controller is electrically decoupled from the voltage converter and from the energy store. The switching and the voltage converter may be fully or partially integrated in the motor controller. In one embodiment, the selector switch switches the circuit configuration of the energy store.

In one development, the voltage converter consists of at least one semiconductor switch, in which case at least one of the semiconductor switches may be configured as part of the switching apparatus.

According to one development, the control device is configured in such a way that an increase of the supply voltage of the motor controller is performed only when a rotational speed threshold of the electric motor is exceeded. The supply voltage of the motor controller may depend on the motor rotational speed, the motor torque, the motor current and/or the time profiles thereof. A motor rotational speed need not be measured in order to ascertain whether a rotational speed threshold is reached and exceeded, but instead a rate of change of a degree of freedom coupled therewith may be measured and the exceeding of a rotational speed or of a rotational speed threshold of a motor may therefore be inferred.

Exemplary embodiments of the invention are explained in more detail below with the aid of the appended figures, in which:

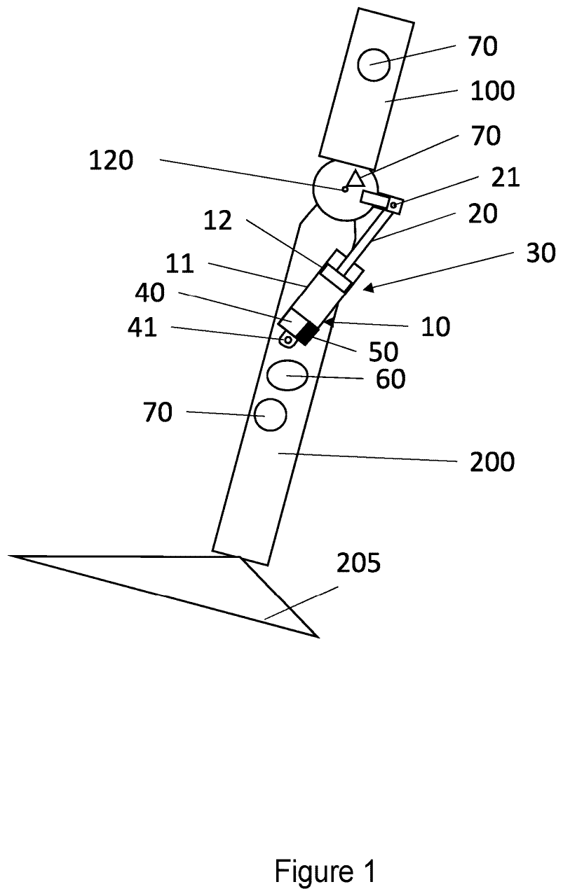

FIG. 1—shows a schematic representation of a prosthesis;

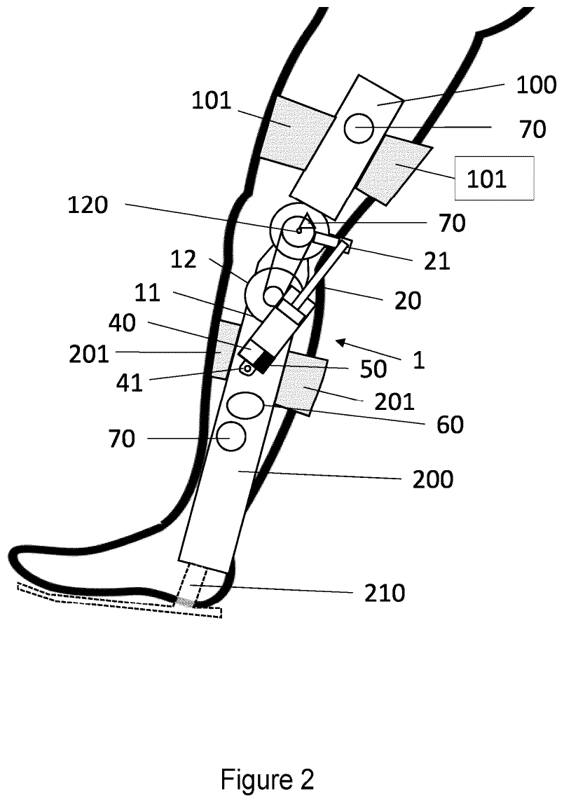

FIG. 2—shows a schematic representation of an orthosis;

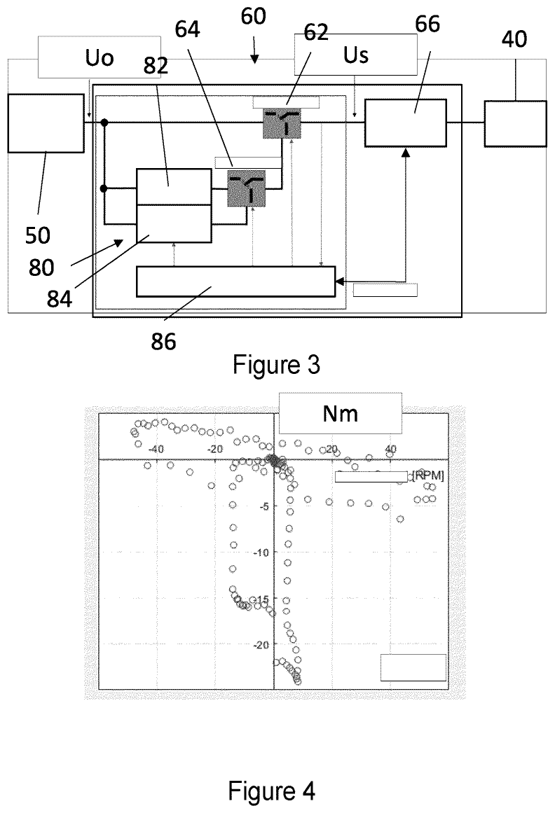

FIG. 3—shows a block circuit diagram of a voltage supply;

FIG. 4—shows a plot representation of gait data;

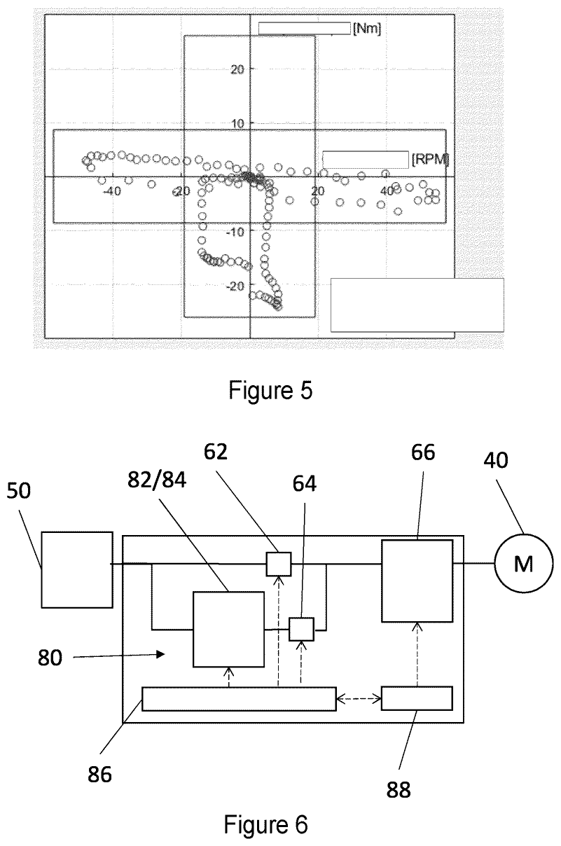

FIG. 5—shows an exemplary block diagram with different working ranges;

FIG. 6—shows an alternative circuit diagram;

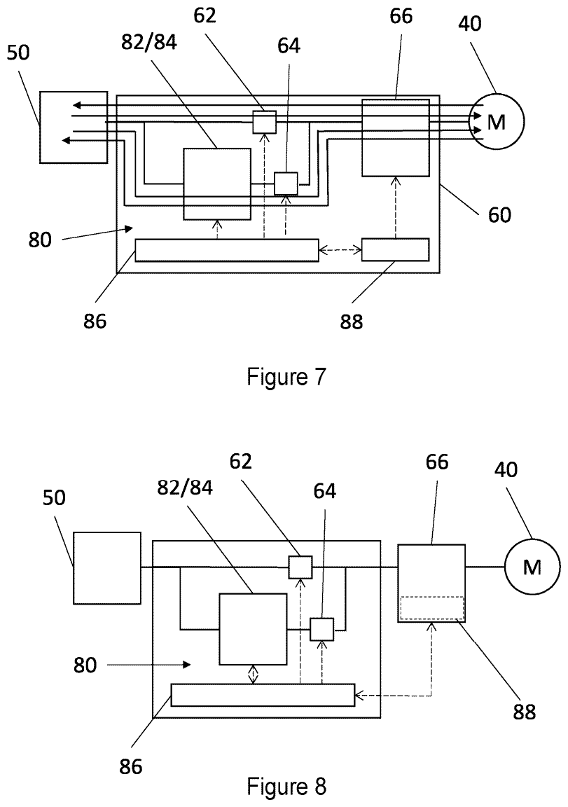

FIG. 7—shows the energy and signal flows in the circuit diagram according to FIG. 6;

FIG. 8—shows a variant of FIG. 6.

A schematic representation of an artificial knee joint is represented in FIG. 1 as part of a prosthesis and in FIG. 2 as part of an orthosis as an orthopedic device. The artificial knee joint has an upper part 100 and a lower part 200, which are mounted pivotably to one another about a pivot axis 120. On the lower part 200, in an embodiment as a prosthesis, a prosthetic foot 205 is arranged on the distal end, and in the embodiment of the artificial knee joint as an orthotic knee joint, as shown in FIG. 2, the lower part 200 is configured as a lower leg rod on which a foot part is not arranged, although an optional foot part 210 (which is represented by the broken line) may be arranged on it. For the case of a KAFO, a foot part 210 on which a foot can be placed is arranged on the lower part 200. In order to produce a pure knee orthosis, however, this may also be omitted. In the embodiment as a prosthetic leg according to FIG. 1, a prosthesis socket or another device for receiving a femoral stump or for fixing to a person is arranged or formed on the upper part 100. In the embodiment according to FIG. 2, the orthosis is fixed to a leg by fastening means 101, 201, which are configured by way of example as straps, shells or the like, in order to fix the orthosis removably to the leg. A further difference between the embodiment according to FIG. 1 and the embodiment according to FIG. 2 is that an alternative drive is provided according to FIG. 2, in which an electric motor 40 is coupled to a pulley, optionally via a gearing. By a V-belt or toothed belt, a flexion or extension of the knee joint may be brought about or assisted, depending on the direction of rotation of the motor. The embodiment having the drive with an electric motor 40 via a mechanical force transmission device and parallel damping by a hydraulic damper may also be employed in a prosthetic knee joint or another prosthetic device.

An actuator 30 as a linearly acting hydraulic motor is arranged between the upper part 100 and the lower part 200. In the exemplary embodiment represented, the hydraulic actuator 30 is configured with a hydraulic chamber or a cylinder 11 that is arranged or formed in a housing or base body 10. A piston 12 is mounted displaceably in the cylinder 11. The piston 12 is displaceable along the longitudinal extent of the cylinder 11 and is fastened to a piston rod 20 that protrudes from the housing or base body 10. The piston 12 subdivides the cylinder 11 into chambers, which are in fluidic communication with one another via a hydraulic line. The base body 10 or the housing may be mounted pivotably to the lower part 200 in order to prevent twisting of the piston 12 during a pivoting movement of the upper part 100 relative to the lower part 200. The end of the piston rod 20 facing away from the piston 12 is fastened to the upper part 100, in the exemplary embodiment represented on a bracket in order to increase the distance from the pivot axis 120. During a flexion, the piston 12 is pressed downward so that the volume of a flexion chamber is reduced and the volume of an extension chamber is correspondingly increased, reduced by the volume of the piston rod 20 that is inserted. Arranged in the housing 10 in order to generate a pressure inside one of the chambers, there is an electric motor 40 which drives in a pump (not represented) in order to apply a pressure to the hydraulic fluid inside one of the two chambers and thereby move the piston 12 in one direction or the other inside the cylinder. This brings about a flexion movement or an extension movement of the orthopedic device in the form of the prosthetic leg.

In the exemplary embodiment represented, the actuator 30 is fixed to the lower part 200 by a fastening device 41. A sensor 70 for recording the spatial orientation of the lower part 200, or of the upper part 100, is respectively arranged both on the upper part 100 and on the lower part 200. By the sensor 70, which may for example be configured as an IMU (inertial measurement unit), the spatial angle or the absolute angle with respect to a fixed spatial orientation, for example the direction of gravity, is ascertained during the use of the artificial knee joint. Instead of an IMU, the sensor 70 may also record other status data, in particular status data that relate to the artificial knee joint. Positions, angular settings, speeds, accelerations, forces and profiles, or changes thereof, are in particular recorded as status data. The ascertained spatial angle of the upper part 100 and/or of the lower part 200, or another status variable, is compared with a threshold angle. When a threshold value that is stored in a controller for the respective sensor value, or a quantity derived therefrom, is reached or exceeded, an actuator is activated or deactivated in order to modify the flow resistance in the actuator 30.

The actuator 30 in an artificial knee joint is used to moderate a flexion movement and an extension movement in order to generate or assist a suitable or desired movement sequence. An extension movement is optionally assisted and advantageously braked shortly before reaching a maximum extension, in order to avoid a hard stop. A flexion movement is braked or suppressed in the stance phase and in the swing phase in order to ensure limitation of the bending. In order to be able to drive the electric motor 40, an energy store 50, particularly in the form of an accumulator, is likewise arranged in the actuator 30. The energy store 50 may also be arranged elsewhere in the orthopedic device, where more space is available or this is deemed to be advantageous because of the weight distribution.

As an alternative to the embodiment of the actuator 30 as a hydraulic motor with an electric motor 40 and a pump, the actuator 30 may also have direct mechanical coupling of the electric motor 40 to the upper part 100 and to the lower part 200 via a force transmission device, for example by a spindle drive, so that instead of a piston rod 20 a spindle rod is inserted into or retracted from the housing 11 by the rotation of a spindle nut, which is driven by the electric motor 40. The actuator 30 is in one embodiment coupled via a gearing device to the upper part 100 and the lower part 200, for example via a planetary gear, in order to displace the upper part 100 relative to the lower part 200. As an alternative to an embodiment of the orthopedic device as an orthosis or prosthesis or an exoskeleton of the lower extremity, it may also be configured as an orthosis or prosthesis or an exoskeleton of the upper extremity.

Other orthopedic devices having two mutually displaceable components, the relative movements of which with respect to one another are influenced by an electric motor, also make use of the invention.

Furthermore, a control device 60 and at least one angle recording device 70 as a sensor are arranged on the orthopedic device. The angle recording device 70 records the angle between the upper part 100 and the lower part 200 and is configured, for example, as a direct angle sensor that detects the angle directly. Alternatively, the angle between the upper part 100 and the lower part 200 may also be ascertained by evaluating the sensor data of the spatial location sensors 70. Both methods may also be used simultaneously or complementarily. All the sensors arranged on the orthopedic device are coupled to a control device 60, and their sensor values are used as the basis for controlling the actuator 30. The actuator 30 is driven on the basis of the sensor data, in particular the spatial locations and/or angular positions, as well as position data and data relating to the loading, orientation, acceleration and/or deformation of further components, for example in order to activate, deactivate or moderate the electric motor 40, for example in order to reduce or increase a pivoting resistance, to limit an end stop and/or to generate or assist a relative movement between the upper part 100 and the lower part 200.

FIG. 3 represents a circuit diagram of the voltage supply of the electric motor 40 of the energy store 50. Arranged between the electric motor 40 and the energy store 50, there is the control device 60, which has a plurality of components in order to supply the electric motor 40 with the respectively required or desired supply voltage Us. The output voltage Uo is output by the energy store 50 and initially supplied unmodified to the motor controller 66. Inside the control device 60, there is a first selector switch 62 and a direct motor controller 66 which, on the basis of data, in particular sensor data, ascertains and determines the supply voltage Us which is or needs to be supplied to the electric motor 40 in order to achieve an optimal outcome. In an initial setting, the first selector switch 62 is in a closed setting in which the output voltage Uo of the energy store 50 is supplied directly as a supply voltage Us to the motor controller 66. Therefore, with the output voltage Uo as the supply voltage Us, the maximum motor currents can be supplied via the motor controller 66 to the electric motor 40 at a low motor voltage.

If it is detected by a computer via the motor controller 66 or another evaluation device that an increased rotational speed of the electric motor 40 is required, for example in order to pivot the lower part 200 rapidly relative to the upper part 100 in a relaxed state, the motor controller 66 sends a corresponding control signal to a voltage regulator 86. The voltage regulator 86 knows the switch setting of the first selector switch 62 and sends a corresponding instruction to the first selector switch 62 and a first voltage converter 82, which is configured as a step-up converter. The step-up converter 82 transforms the output voltage Uo of the energy store 50 to a higher level and then forwards this intermediate-circuit voltage as a supply voltage Us via the motor controller 66 to the electric motor 40. The control signal of the motor controller 66 therefore delivers the instruction to adjust the respectively needed supply voltage Us. The switching to the supply voltage Us with a higher voltage in comparison with the output voltage Uo takes place with the first selector switch 62, which now provides the output voltage of the step-up converter 82 as Us. The switching process is advantageously facilitated by a dynamically adjustable step-up converter 82. Soft switching between the various voltage sources is therefore possible. Moreover, losses inside the step-up converter 82 can be reduced. Losses are in particular reduced by the output voltage Uo being capable of following the increased voltage requirement. The output voltage level is raised only as much as is needed. With an increased supply voltage Us in comparison with the output voltage Uo, the maximum motor currents are reduced so that, although the electric motor 40 can achieve a higher rotational speed, it only provides a lower torque.

In the embodiment represented, the voltage converter is configured bidirectionally and, besides the step-up converter 82, also has a step-down converter 84 with which it is possible to step down increased voltages or draw energy from the movement, which is received via the motor 40, and feed it back to the energy store 50. For this purpose, the second selector switch 64 is assigned to the voltage controller 86 so that, in corresponding load states, movement states and energy states, the control device 60 drives the two selector switches 62, 64 via the motor controller 66 and the voltage regulator 86 and, according to requirements, increases the output voltage Uo or resets it to the initial level, or feeds electrical energy back from the motor 40 in generator operation to the energy store 50 for recharging.

FIG. 4 represents an exemplary profile of motor-generated torque in the knee axis as a function of the resulting rotational speed during a step. The individual circles represent the measured points. For each step, the respective initial torque of the electric motor in Nm is plotted as a function of the rotational speed per minute in rpm. It shows a correlation of a comparatively high torque of up to minus 25 Nm, at the low rotational speed of between 10 and minus 15 rpm, and a high adjustment speed of from minus 50 rpm to plus 60 rpm at an initial torque of not more than plus/minus 5 newton-meters. The large spread between the ranges with a high torque and low rotational speeds and the ranges with a low torque and high rotational speeds is characteristic of the use of an active drive of a knee prosthesis. In an electric drive with an electric motor, there is a proportionality between the required supply voltage Us and the rotational speed of the drive, as well as a proportionality between the torque of the drive and its motor current. The maximum values of the current and the supply voltage Us therefore determine on the one hand the maximum torque and on the other hand the maximum rotational speed of the drive. An energy store is conventionally constructed from a plurality of cells of an accumulator system. With a given number of cells, either the overall system may be optimized for high currents by a parallel circuit of a plurality of cells or it is optimized for high voltages by a series interconnection of the cells. If both ranges, that is to say high torques and high rotational speeds, are equally intended to be achieved, further cells need to be added. This increases the volume and therefore also the weight of the energy store.

FIG. 5 represents an exemplary plot of gait data of an active knee prosthesis with an energy supply corresponding to a circuit according to FIG. 3. The motor-generated torque in the knee axis is represented as a function of the resulting rotational speed during a step. The individual circles represent the measured points. It indicates the two working ranges, which are switched between by the selector switches 62, 64. The vertical rectangle symbolizes the range with a supply voltage Us that corresponds to the output voltage Uo, that is to say it allows a comparatively high torque with a comparatively low rotational speed. The horizontally oriented rectangle is the range in which the electric motor 40 operates with an increased supply voltage Us in relation to the output voltage Uo so that a higher maximum rotational speed is possible, albeit with a reduced maximum torque. The vertically oriented rectangle indicates the operating range in which a maximum output current can be supplied to the electric motor 40 at the output voltage Uo, so that the range of the high torques is covered. The horizontally oriented working range represents the voltage range increased by the voltage regulator 86 and the step-up converter 82 with a reduced maximum current. High rotational speeds at comparatively low torques can be covered. The overlap region of the two ranges is advantageously covered by the vertical working range with an output voltage Uo transmitted directly without the interposition of a converter 82.

The voltage converters 82, 84 are dynamically adjustable, and during phases with an increased motor voltage the step-down converter 84 converts electrical energy generated from the movement in generator operation of the electric motor 40 into the corresponding voltage for charging the energy store 50. In phases with motor voltages Us less than the output voltage Uo and a deactivated voltage converter 84, when there is a direct connection to the energy store 50, the electric motor 40 can charge the energy store 50 via the motor controller 66. The step-up conversion also allows the behavior of the orthopedic device in respect of the achievable rotational speeds to remain the same even in the event of a decreasing voltage with a draining energy store 50.

FIG. 6 represents a diagram of a motor controller as an alternative to the motor controller according to FIG. 3, in which the same references denote the same constituent parts. FIG. 7 represents the respective energy flows by solid arrows. Energy is supplied to the motor 40 from the energy store 50, or in generator operation of the motor 40 energy is supplied to the energy store 50. In this embodiment as well, the control device 60 has a first selector switch 62 and a second selector switch 64, the second selector switch 64 being optional. The motor controller 66 is regulated, or controlled, by a motor regulator 88 which is in turn connected to and exchanges control signals with the voltage regulator 86. The exchange of the control signals is represented by the dashed arrows. The motor regulator 88 also influences the motor controller 66.

The top arrow according to FIG. 7 shows the energy flow in generator operation or reverse operation, in which the motor 40 is driven. The electrical energy generated in the motor 40, for example during braking of a prosthetic or orthotic joint, is returned to the energy store 50. If the current is high and the voltage is low, the transmission takes place without the interposition of a voltage converter. Conversely, in the conventional operation which is indicated by the second arrow from the top, the energy is supplied to the motor 40 directly from the energy store 50 without modification, optionally with step-down conversion, so that in the event of a high current a comparatively low supply voltage is applied to the motor 40.

The third arrow from the top shows the energy flow of a drive in which a higher or increased supply voltage or motor voltage is needed for the motor 40. The electrical energy is conveyed from the energy store 50 through the control device 60 to the corresponding voltage converter 84 in order to generate an increased motor voltage in forward operation. Conversely, in backward operation with an increased motor voltage, which is represented by the bottom arrow, the electrical energy generated by the motor 40 in generator operation is conveyed through the step-down converter 82 and then transmitted to the energy store 50.

FIG. 8 represents a variant of FIG. 6, in which the motor regulator 88 is configured as an integral part of the motor controller 66.

In principle, the energy supply may also be completely separated from the motor controller 66 or from the motor 40.

1. An orthopedic device, comprising:
an upper part;
a lower part mounted displaceably on the upper part; and
an actuator configured to displace the lower part relative to the upper part, wherein the lower part is coupled to the actuator, and wherein the actuator comprises
an electric motor,
an energy store,
a control device, and
a first selector switch is arranged between the energy store and the electric motor, wherein the first selector is coupled to the control device, wherein the first selector is configured to switch a supply voltage of a motor controller of the electric motor between an output voltage of the energy store and a voltage converter with a supply voltage that is modified in relation to the output voltage of the energy store.

2. The orthopedic device as claimed in claim 1, wherein the output voltage of the voltage converter is dynamically adjustable.

3. The orthopedic device as claimed in claim 1, wherein the voltage converter is configured to convert both up and down, or wherein the voltage converter comprises a step-up converter and a step-down converter.

4. The orthopedic device as claimed in claim 3, further comprising a second selector switch for switching between the step-up converter and the step-down converter, wherein the second selector switch is coupled to the control device.

5. The orthopedic device as claimed in claim 1, wherein in an initial setting of the first selector switch, the output voltage of the energy store is applied as a supply voltage of the motor controller of the electric motor.

6. The orthopedic device as claimed in claim 1 wherein the first selector switch is configured as part of the voltage converter.

7. The orthopedic device as claimed in claim 1 wherein the voltage converter comprises one or more semiconductor switches, and wherein at least one of the one or more semiconductor switches is configured as the first a selector switch.

8. The orthopedic device as claimed in claim 1 wherein the control device is configured such that an increase of the supply voltage of the motor controller is performed only when a rotational speed threshold of the electric motor is exceeded.

9. The orthopedic device as claimed in claim 1 wherein the supply voltage of the motor controller or of the motor is dependent upon a motor rotational speed, a motor torque, a motor current and/or time profiles thereof.

10. The orthopedic device as claimed in claim 4 wherein the second selector switch is configured as part of the voltage converter.

11. The orthopedic device as claimed in claim 4 wherein the voltage converter comprises one or more semiconductor switches, and wherein at least one of the one or more semiconductor switches is configured as the second selector switch.
